(19) United States
(12) Patent Application Publication
(10) Pub. No.: US 2010/0030227 A1
(43) Pub. Date: Feb. 4, 2010

(54) MEDICAL LEAD IMPLANTATION

(75) Inventors: John E. Kast, Hugo, MN (US); Michael J. Baade, Zimmerman, MN (US)

Correspondence Address: SHUMAKER & SIEFFERT , P.A, 1625 RADIO DRIVE , SUITE 300, WOODBURY, MN, 55125, US

(73) Assignee: Medtronic, Inc.

(21) Appl. No.: 12/183,224

(22) Filed: Jul. 31, 2008

(51) Int. Cl. A61B 17/00

(52) U.S. Cl. 606/129

(57) ABSTRACT

A medical lead introducer comprises a shank, a carrier structure on the shank configured to engage a mating carrier structure of a medical lead during a lead introduction procedure, and a blunt dissection element located on a distal end of the shank. The blunt dissection element is configured to shield at least a distal portion of the medical lead when the medical lead is engaged by the carrier structure during the lead introduction procedure. In some embodiments, the medical lead introducer may be part of a kit including the medical lead.

### TECHNICAL FIELD

The disclosure relates to medical devices and, more particularly, to implantation of implantable medical devices such as leads.

### BACKGROUND

Electrical stimulation systems may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. An electrical stimulation system typically includes one or more implantable medical leads coupled to an external or implantable electrical stimulator.

The implantable medical lead may be percutaneously or surgically implanted in a patient on a temporary or permanent basis such that at least one stimulation electrode is positioned proximate to a target stimulation site. The target stimulation site may be, for example, a nerve or other tissue site, such as a spinal cord, pelvic nerve, pudendal nerve, occipital nerves, stomach, bladder, or within a brain or other organ of a patient, or within a muscle or muscle group of a patient. The lead may be coupled to a stimulation generator such that the one or more electrodes located proximate to the target stimulation site may deliver electrical stimulation therapy to the target stimulation site in the form of electrical signals.

Percutaneous leads and catheters are often preferred over surgically implanted leads because percutaneously implanted leads are implanted in a less invasive manner. For example, in order to implant percutaneous leads, an incision is made to ease the introduction of an introducer, such as a percutaneous needle. The needle is inserted through the incision and positioned to access a target tissue site. The lead is then inserted through the needle and positioned to adjacent the target tissue site. After the lead has been properly positioned, the needle is withdrawn and the lead is connected to a stimulation device. The stimulation device is typically implanted just below the patient's skin.

### SUMMARY

In general, the disclosure is directed to techniques for implanting a medical lead proximate to a target tissue site within a patient. The disclosed techniques make use of a medical lead introducer including a carrier structure configured to carry a distal section of a lead to a target tissue site. The carrier may include a tab or other structure that engages a portion of the lead during implantation. The lead introducer may include a blunt dissection element at its distal end. A blunt dissection element is a tapered feature at the front the lead introducer that facilitates tunneling through patient tissue. The medical lead introducer carries a medical lead while tunneling through patient tissue to locate the medical lead proximate to the target tissue site. The blunt dissection element may shield at least a portion of the frontal area of the medical lead when tunneling through patient tissue. Once the lead is positioned proximate to the target tissue site, the medical lead is released from the lead introducer and the lead introducer is retracted, leaving the distal end of the lead within the patient proximate a target tissue site.

In one embodiment, a medical lead introducer comprises a shank, a carrier structure on the shank configured to engage a mating carrier structure of a medical lead during a lead introduction procedure, and a blunt dissection element located on a distal end of the shank. The blunt dissection element is configured to shield at least a distal portion of the medical lead when the medical lead is engaged by the carrier structure during the lead introduction procedure.

In another embodiment, a method for introducing a medical lead comprises inserting an assembly into a patient. The assembly comprises a medical lead introducer including a blunt dissection element located at a distal end of the medical lead introducer, and a medical lead attached to the medical lead introducer. The method also includes advancing the assembly through tissue of the patient to locate at least a portion of the medical lead proximate to a target tissue site. Advancing the assembly causes the blunt dissection element to tunnel through tissue of the patient. The method further includes detaching the medical lead from the medical lead introducer and retracting the medical lead introducer leaving a stimulation electrode of the medical lead within the patient proximate to the target tissue site.

Another embodiment is directed to a kit to facilitate implantation of a medical lead into a patient comprising the medical lead and a medical lead introducer. The medical lead introducer comprises a shank, a carrier structure on the shank configured to engage a mating carrier structure of the medical lead during a lead introduction procedure, and a blunt dissection element located on a distal end of the shank. The blunt dissection element is configured to shield the medical lead when the medical lead is held by the carrier structure during a lead introduction procedure.

The details of one or more aspects of the disclosure are set forth in the accompanying drawings and the description below. Other features, objects, and advantages of the disclosed techniques will be apparent from the description and drawings, and from the claims.

### BRIEF DESCRIPTION OF DRAWINGS

FIGS. 1A-1B illustrate a medical lead introducer including a blunt dissection element and a tab configured to engage a distal end of a medical lead.

FIGS. 2A-2B illustrate a medical lead configured for insertion within a patient using the medical lead introducer of FIGS. 1A-1B.

FIG. 3 illustrates a kit including a medical lead and a medical lead introducer packaged in a sterile container.

FIGS. 4A-4C illustrate a medical lead introducer including a blunt dissection element and sidewalls that increase the stiffness relative to the medical lead introducer of FIGS. 1A-1B.

FIGS. 5A-5C illustrate a medical lead introducer including a blunt dissection element and a retractable tab configured to engage a distal end of a medical lead.

FIGS. 6A-6B illustrate a patient and a therapy system implanted in the patient, wherein the therapy system includes an electrical stimulator coupled to two stimulation leads for occipital nerve stimulation.

FIG. 7 illustrates a kit including a medical lead and a medical lead introducer with an different carrier structure relative to the kit shown in FIG. 3.

FIG. 8 is a flowchart that illustrates techniques for introducing a medical lead.

### DETAILED DESCRIPTION

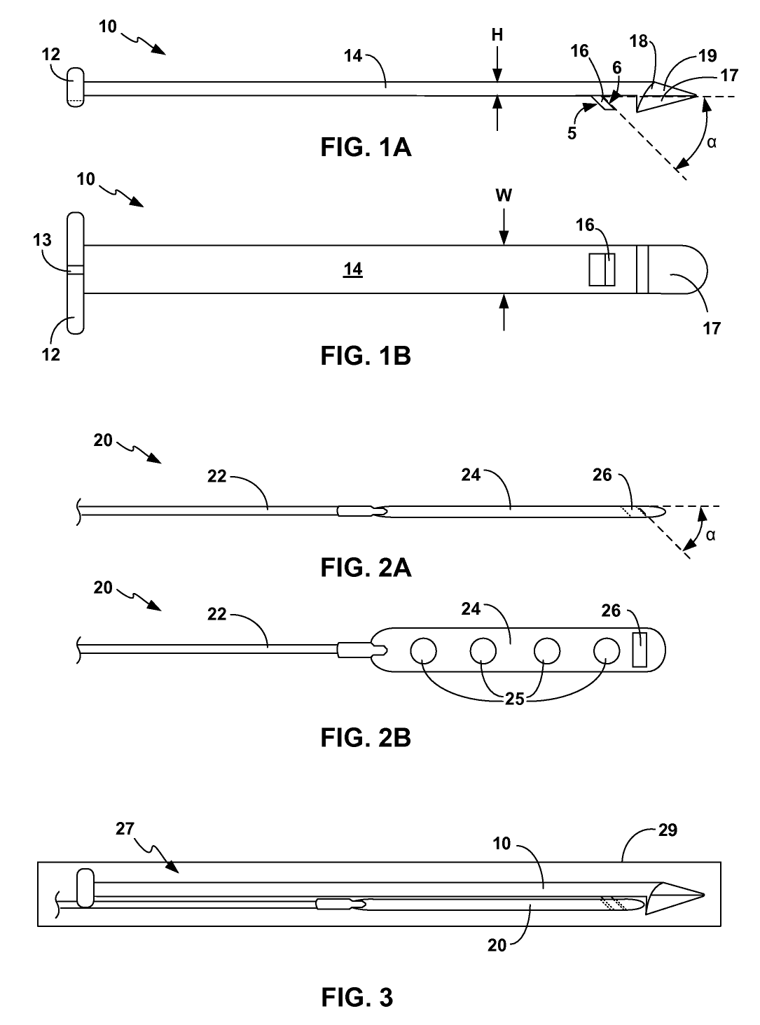

FIGS. 1A-1B illustrate medical lead introducer 10, which includes blunt dissection element 18. FIGS. 2A-2B illustrate medical lead 20, which is configured for insertion within a patient using the medical lead introducer 10. FIG. 3 illustrates a kit including medical lead 20 and medical lead introducer 10 packaged in sterile container 29. Medical lead introducer 10 is configured such that one side of medical lead 20 is exposed to patient tissue during a lead introduction procedure. Lead introducer 10 facilitates the positioning of medical lead 20 proximate a target tissue site within a patient simultaneously with the blunt dissection of patient tissue.

In the example of FIGS. 2A and 2B, medical lead 20 is a paddle lead including one or more electrodes 25 on paddle 24 to deliver stimulation therapy to therapy region within a patient. Medical lead 20 also includes lead body 22, which includes insulated conductors in electrical communication with electrodes 25. In other examples, the lead may be an axial lead, e.g., with ring or segmented electrodes. A proximal end of medical lead 20 is configured to be electrically and mechanically connected to an electrical stimulation therapy delivery device to deliver stimulation therapy to a patient via electrodes 25. Electrodes 25 may also be used as sensing electrodes to sense one or patient parameters, including, but not limited to, patient parameters related to a patient response to stimulation. In addition, medical lead 20 may include fluoroscopic elements to allow a clinician to more easily determine an orientation and position of medical lead 20 using fluoroscopy during implantation.

Medical lead introducer 10 is configured to implant medical lead 20 proximate a target tissue site within a patient. Medical lead introducer 10 includes shank 14 and blunt dissection element 18, which is fixed to the distal end of shank 14. Blunt dissection element 18 may have a tip radius of approximately 0.020 inches to 0.075 inches to dissect subcutaneous tissue. The overall length of the introducer may range from approximately 3 inches to 8 inches with a width of approximately 0.075 inches to 0.500 inches.

Lead introducer 10 includes handle 12 on the proximal end of lead introducer 10. Handle 12 allows a clinician to apply a significant force to lead introducer 10 in order to tunnel through tissue of a patient using blunt dissection element 18. Generally, the profile of medical lead introducer 10 should be kept to a minimum to limit the size of a tunnel created within a patient when implanting medical lead 20 with medical lead introducer 10. Limiting the size of the tunnel may not only reduce patient trauma associated within implantation, but may also reduce lead migration after implantation.

Shank 14 has a rectangular cross section, although in other examples different cross-sectional shapes may also used. For example, the width W of shank 14 may be at least three times greater than the height H of shank 14. Generally, the width of shank 14 may be about equal to the width of paddle 24. The cross section of medical lead introducer 10 may cause shank 14 to have greater flexibility about its height and limited side-to-side flexibility. The uneven flexibility provided by shank 14 may improve the steerability of lead introducer 10 when tunneling through tissue of a patient by substantially constraining the bending of shank 14 to be within a single plane. In different examples, lead introducer 10 may be may substantially stiff such that it will not bend during a blunt dissection procedure.

In the example of FIGS. 1A and 1B, medical lead introducer 10 also includes tab 16, which extends from shank 14. Tab 16 is configured to engage through-hole 26 of medical lead 20 to hold medical lead 20 during a lead introduction procedure. In this manner, tab 16 serves as a carrier structure, whereas through-hole 26 serves as a mating carrier structure. Through-hole 26 is located at the distal end of medical lead 20 in paddle 26. In other examples, paddle 26 may include a detent as a mating carrier structure to be engaged by tab 16 in place of through-hole 26. In other examples, carrier structures may include additional tabs similar to tab 16 to engage multiple depressions on a medical lead.

Tab 16 extends at a forward angle α relative to the insertion direction of lead introducer 10. Likewise, through-hole 26 passes through paddle 24 at about the same angle α. As examples, the angle α may be between 10 and 80 degrees, between 30 and 60 degrees, or may be about 45 degrees. In some embodiments, distal surface 6 of tab 16 may be at different angle as compared to proximal surface 5 relative to the insertion direction of lead introducer 10. For example, distal surface 6 may be at a larger angle than proximal surface 5 of lead introducer 10. This may increase the strength of tab 16 for a given angle of the proximal surface 5 as a smaller angle of the proximal surface 5 may make it easier to release lead 20 from lead introducer 10. As examples, distal surface 6 may be at an angle of between 5 and 75 degrees greater than the angle of proximal surface 5, at an angle of between 15 and 60 degrees greater than the angle of proximal surface 5 or at an angle of about 20 degrees greater than the angle of proximal surface 5.

Detent 13 also may be provided to help secure medical lead 20 during a lead introduction procedure. Detent 13 is located on handle 12, and is configured to secure lead body 22 as shown in FIG. 3. For example, detent 13 may provide a snap-fit interface with lead body 22. This snap-fit interface may assist in keeping lead body 22 in line with lead introducer tool 10 and may also hold through-hole 26 in paddle 24 of lead 20 in engagement with tab 16.

Lead introducer 10 is inserted as part of an assembly also including lead 20 into the tissue of a patient. Tab 16 extends from shank 14 and is angled towards the distal end of lead introducer 10, i.e., towards blunt dissection element 18. Likewise, through-hole 26 has a similar angled configuration to mate with tab 16. As a clinician forces lead introducer 10 through patient tissue, friction of patient tissue pulls on lead 20 including paddle 24. The angled configuration of tab 16 and through-hole 26 holds tab 16 in engagement with paddle 24. The clinician continues to force introducer 10 in a forward direction through patient tissue until electrodes 25 are positioned adjacent a target tissue site.

After advancing lead 20 to the desired location, the clinician withdraws introducer 10. The angled configuration of tab 16 and through-hole 26 allows tab 61 to withdraw from through-hole 26 and introducer 10 to slide out over lead 20 without significantly disturbing placement of lead 20. An important feature of lead introducer 10 is that it does not encompass lead 20 during lead placement within a patient, i.e., at least one side of lead 20 is exposed to patient tissue during implantation. This allows lead 20 to be implanted simultaneously while tunneling through patient tissue. It also facilitates implantation of leads that are permanently fixed to a stimulation device since the introducer does not need to slide off the proximate end of the lead. While the specific example of tab 16 and through-hole 26 are suitable as a carrier structure and mating carrier structure respectively, many other structures may also be used for a lead introducer that does not encompass the lead during lead placement within a patient.

Blunt dissection element 18 may have a tapered tip to facilitate blunt dissection through tissue of a patient. As best shown in FIG. 3, blunt dissection element 18 has a frontal area that extends beyond a frontal area of shank 14. As referred to herein, a frontal area is the two-dimensional area in the geometric plane that is perpendicular to the insertion direction. In this manner, blunt dissection element 18 provides a frontal area that shields medical lead 20 when medical lead 20 is held by medical lead introducer 10 during a lead introduction procedure. Because blunt dissection element 18 is not centered on the distal end of shank 14, the insertion force applied by a clinician to handle 12 does not inherently balance with the tunneling force applied to blunt dissection element 18 by tissue of a patient. Instead, the off-center position of shank 14 relative to blunt dissection element 18 biases lead introducer 10 down in the direction of lead 10. For this reason, blunt dissection element 18 is asymmetrical to balance the insertion force against the blunt dissection force. This limits bending of the medical lead introducer resulting from the combination of the insertion force and the blunt dissection force. For example, blunt dissection element 18 may defined a surface 17 that is proximate to the side of shank 16 that includes tab 16 and surface 19, which opposes the first surface 17. In order to balance the insertion force against the blunt dissection force, the frontal area of surface 17 may be greater than the frontal area of the surface 19.

Medical lead introducer 10 may be made of any material suitable for facilitating implantation of a medical lead. In addition, medical lead introducer 10 may include fluoroscopic elements to allow a clinician to more easily determine an orientation and position of the lead introducer 10 using fluoroscopy during implantation of a medical lead. For example, medical lead introducer 10 may be made from stainless steel, titanium, polyester, polyurethane, silicone, polysulfone and/or polycarbonate plastic, or other biocompatible materials. In some instances, all or a portion of lead introducer 10 may be coated, e.g., with Polytetrafluoroethylene (PTFE), to reduce friction with a patient's tissue during a lead introduction procedure.

As shown in FIG. 3, medical lead introducer 10 may come packaged as a kit including medical lead 20 packaged in sterile container 29. As examples, sterile container 29 may be a flexible plastic enclosure, pouch, Tyvek® sterilization bag, foil packaging or other suitable sterile container. In such an example, lead introducer 10 may be disposable after implantation of lead 20. In other examples, lead introducer 10 may be reused to implant multiple leads.

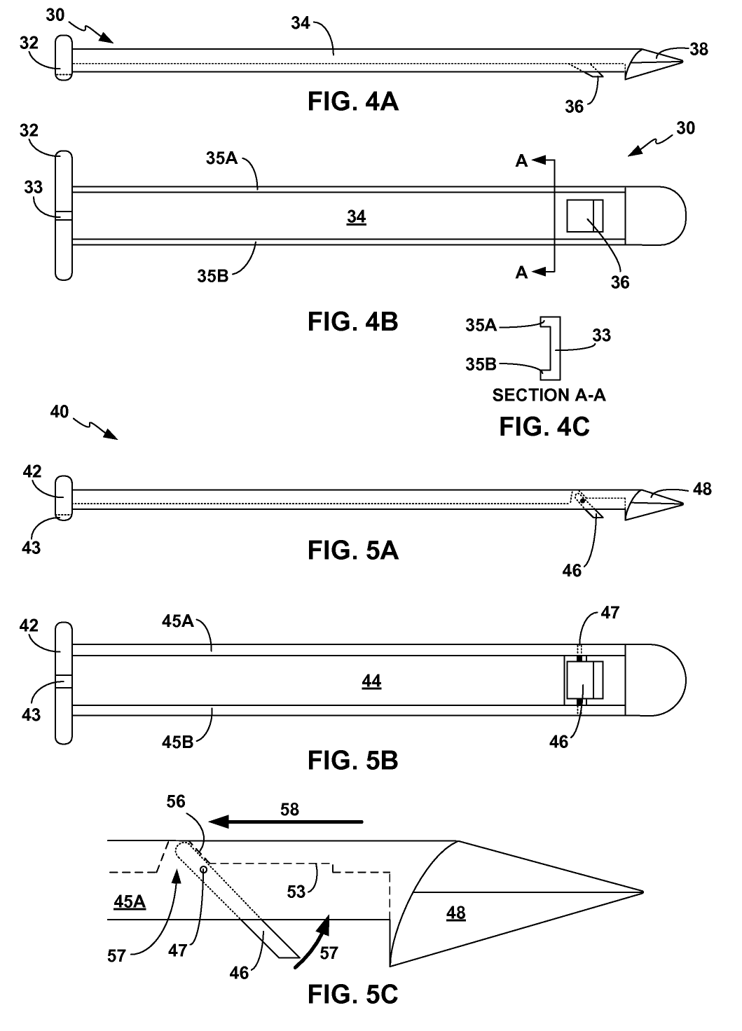

FIGS. 4A-4C illustrate another example of a medical lead introducer 30. Medical lead introducer 30 is similar to medical lead introducer 10 except that shank 34 includes a center element 33 and two side elements 35A and 35B (collectively referred to as side elements 35) extending from the edges of the center element, giving shank 34 a C-shaped cross section. Like medical lead introducer 10, medical lead introducer 30 is suitable for implanting medical lead 20. Except for the inclusion of side elements 35, lead introducer 30 generally conforms to lead introducer 10. For brevity, details discussed with respect to medical lead introducer 10 that are the same for lead introducer 30 are discussed in limited detail or not at all with respect to medical lead introducer 30.

Medical lead introducer 30 is configured to implant a medical lead proximate to a target tissue site within a patient. Medical lead introducer 30 includes shank 34 and blunt dissection element 38, which is fixed to the distal end of shank 34. Lead introducer 30 includes handle 32 on the proximal end of lead introducer 30. Generally, the profile of medical lead introducer 30 should be kept to a minimum to limit the size of a tunnel created within a patient when implanting medical lead with medical lead introducer 30.

Shank 34 includes side elements 35 that give shank 34 a C-shaped cross section. Generally, the width of shank 34 inside of side elements 35 will be about equal to the width of paddle of a paddle lead. Side elements 35 limit the flexibility of shank 34 which may make it easier to apply a force to handle 32 and tunnel through tissue of a patient without bending shank 34 as compared to lead introducer 10. Side elements 35 may also be configured to provide a slight friction fit with a paddle lead to help retain the lead during a lead insertion procedure.

Medical lead introducer 30 also includes tab 36, which extends from the center element of shank 34 in the same direction that side elements 35 extend from the center element of shank 34. Tab 36 is configured to engage a detent, such as a recess or through-hole, of medical lead to hold medical lead during a lead introduction procedure. Medical lead introducer 30 also includes detent 33, which also helps secure a medical lead body during a lead introduction procedure.

Blunt dissection element 38 has a tapered tip to facilitate blunt dissection through tissue of a patient. Blunt dissection element 38 has a frontal area that extends beyond a frontal area of shank 34 to shield a medical lead when medical lead is held by medical lead introducer 30 during a lead introduction procedure. Blunt dissection element 38 is asymmetrical to balance the insertion force against the blunt dissection force.

Medical lead introducer 30 may be made of any material suitable for facilitating implantation of a medical lead. For example, medical lead introducer 30 may be made from stainless steel, titanium, and/or plastic, or other biocompatible materials.

FIGS. 5A-5C illustrate medical lead introducer 40, which is an alternative to lead introducers 10 and 30. Medical lead introducer 40 is similar to medical lead introducer 30 except that medical lead introducer 40 includes a retractable tab 46. For example, medical lead introducer 40 is also suitable for implanting medical lead 20. Except for retractable tab 46, lead introducer 40 generally conforms to lead introducer 30. For brevity, details discussed with respect to medical lead introducers 10 and 30 that are the same for lead introducer 40 are discussed in limited detail or not at all with respect to medical lead introducer 40.

Medical lead introducer 40 is configured to implant a medical lead proximate to a target tissue site within a patient. Medical lead introducer 40 includes shank 44 and blunt dissection element 48, which is fixed to the distal end of shank 44. Shank 44 includes side elements 45A and 45B that give shank 44 a C-shaped cross section. Lead introducer 40 includes handle 42 on the proximal end of lead introducer 40. Generally, the profile of medical lead introducer 40 should be kept to a minimum to limit the size of a tunnel created within a patient when implanting medical lead with medical lead introducer 40.

Medical lead introducer 40 includes retractable tab 46, which extends from the center element of shank 44 in the same direction that side elements 45 extend from the center element of shank 44. A retractable tab such as tab 46 is a tab that moves towards a lead introducer shank, e.g., shank 44 and away from a lead, for withdrawal of the lead introducer after positioning of the lead. Tab 46 is configured to engage a detent of medical lead to hold medical lead during a lead introduction procedure. As best seen in FIG. 5C, tab 46 is pivotable about pivot point 47. The center portion of shank 44 includes a gap 57 to allow tab 46 room to rotate. When lead introducer 40 is inserted into a patient as part of an implant procedure, the range of motion of tab 46 is limited by the distal surface 56 of gap 57, and tab 46 engages a corresponding detent, such as a recess or through-hole on the medical lead. Medical lead introducer 40 also includes detent 43, which also holds the medical lead body during a lead introduction procedure to help hold tab 46 in the engaged position shown in FIG. 5C. However, when lead introducer is retracted from a patient in direction 58, tab 46 rotates in direction 57 and comes to rest in depression 53. In this manner, tab 46 automatically retracts to release a medical lead when medical lead introducer 40 is withdrawn from a patient. In other embodiments, a wire may be attached to tab 46 and extend to a handle located near the proximal end of medical lead introducer 40. Such a wire may be used to actively cause tab 46 to pivot. For example, such a wire may be routed through a hole in shank 44.

Medical lead introducer 40 may be made of any material suitable for facilitating implantation of a medical lead. For example, medical lead introducer 40 may be made from stainless steel, titanium, and/or plastic, or other biocompatible materials.

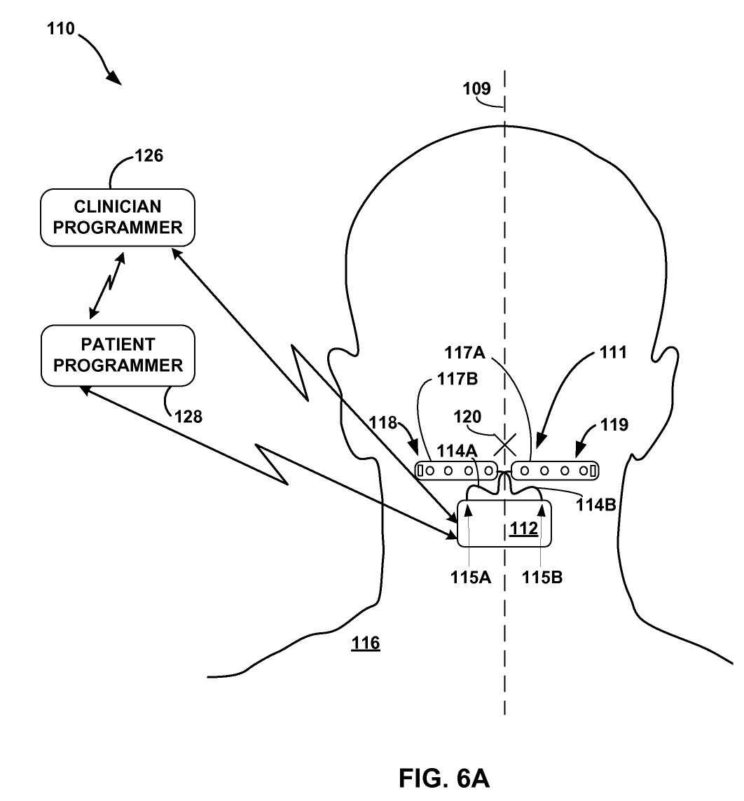

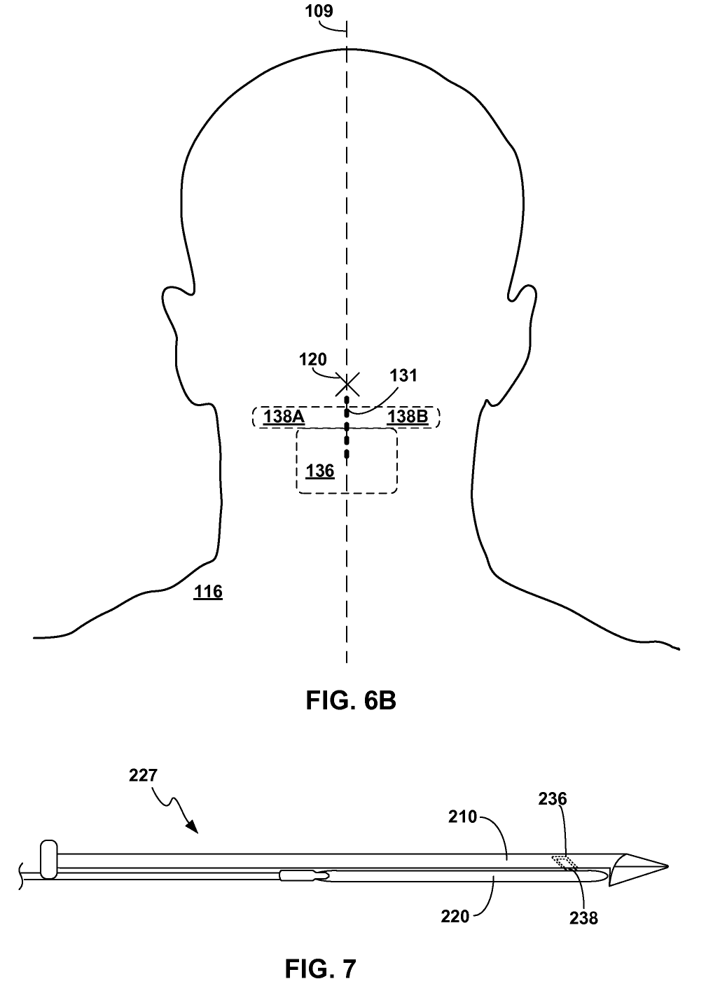

FIG. 6A is a schematic diagram of therapy system 110, which includes an electrical stimulator 12 coupled to stimulation leads 114A, 114B (collectively referred to as “leads 114”). In the example of FIG. 6A, electrical stimulator 112 is implanted in a human patient 116 proximate to an occipital region 111 within patient 116, below inion 120, the craniometric point that is the most prominent point at the occipital protuberance on the back of the head of patient 116.

Paddles 117A, 117B (collectively referred to as “paddles 117”) include electrode sets to deliver stimulation therapy to a therapy region, which generally encompasses occipital nerve sites and trigeminal nerve sites of patient 116. Such nerve sites may include, for example, an occipital nerve (e.g., a greater occipital nerve, lesser occipital nerve, third occipital nerve and suboccipital nerves), a trigeminal nerve, tissue adjacent to the trigeminal or occipital nerves, or a nerve branching from the occipital and/or trigeminal nerves. Thus, reference to an “occipital nerve” or a “trigeminal nerve” throughout the disclosure also may include branches of the occipital and trigeminal nerves, respectively. In addition, the stimulation therapy may be delivered to both an occipital nerve and trigeminal nerve by a single therapy system 110. While paddles 117 include a linear array of electrodes, other examples may utilize paddle electrodes including a two-dimensional array of electrodes.

Electrical stimulator 112 generates a stimulation signal (e.g., in the form of electrical pulses or substantially continuous waveforms). The stimulation signal may be defined by a variety of programmable parameters such as electrode combination, electrode polarity, stimulation voltage amplitude, stimulation current amplitude, stimulation waveform, stimulation pulse width, stimulation pulse frequency, etc.) that is delivered to occipital region 111 by implantable stimulation leads 114, respectively, and more particularly, via stimulation electrodes carried by stimulation leads 114. Electrical stimulator 112 may also be referred to as a pulse or signal generator, or a neurostimulator. In some embodiments, leads 114 may also carry one or more sense electrodes to permit electrical stimulator 112 to sense electrical signals or other sensors to sensor other types of physiological parameters (e.g., pressure, activity, temperature, or the like) from occipital region 111, respectively. In some implementations, for example, such sensed parameters may be recorded for later analysis, e.g., evaluation of stimulation efficacy, or used in the control of stimulation therapy or therapy parameters.

The proximal ends of leads 114 are both electrically and mechanically coupled to separate connection ports 115A, 115B (collectively referred to as “ports 115”) of electrical stimulator 112. Connection ports 115 are each located in a separate connector block within the housing of electrical stimulator 112. The connector blocks including connection ports 115 include terminals at different axial positions within the connector block that mate with contacts at different axial positions at proximal ends of leads 114. The connection between leads 114 and connection ports 115 also includes fluid seals to prevent undesirable electrical discharge. In different embodiments, leads 114 may be removed from connection ports 115 by a clinician if desired. For example, the removable connection may be a pressure or snap-fit, e.g., with a spring contacts. In other embodiments, leads 114 may be fixed to connection ports 115 such that simply pulling on leads 114 will not release them from connection ports 115. Examples of fixed connections include solder connections, set screws or other techniques.

In any event, conductors disposed in the lead body of each of leads 114 electrically connect stimulation electrodes (and sense electrodes, if present) adjacent to the distal ends of leads 114 to electrical stimulator 112. Connection ports 115 are located at least approximately a third of the length of the housing of electrical stimulator 112 apart from each other. For example, if the width of the housing is X, connection ports 115 are located at least ⅓*X apart from one another.

In the example of therapy system 110 shown in FIG. 6A, target tissue sites 118 and 119 are located within the patient's head or neck (e.g., proximate to one or more occipital nerve) and on opposite sides of midline 109 of patient 116. Midline 109 is a schematic representation of the line that divides patient 116 into about equal and symmetrical left and right halves. Delivering therapy to two target tissue sites, such as sites 118 and 119, may be used to deliver therapy to two nerve branches that branch from the same nerve. Nerves may branch into left and right branches that extend to opposite sides of midline 9, and therapy is delivered to two nerve branches on opposite sides of midline 9 (such as at target tissue sites 118 and 119). Stimulation of two nerve branches on opposite sides of midline 9 may be referred to as bilateral stimulation. However, bilateral stimulation may also refer to stimulation of any two regions of patient 116 either sequentially or simultaneously. Delivering therapy after nerves branch, e.g., closer to the nerve endings, may allow more targeted therapy delivery with fewer side effects. Therapy may also be delivered unilaterally to sites 118, 119. For example, stimulation therapy may be delivered to site 118 by paddle 117B simultaneously or alternately with stimulation of site 119 by paddle 117A. In addition therapy may be delivered using an electrode set including at least one electrode from both paddle 117A and 117B.

Stimulation of the occipital region 111 (i.e., in regions of patient 116 proximate to occipital nerves, a trigeminal nerve or other cranial sites) may help alleviate pain associated with, for example, chronic migraines, cervicogenic headaches, occipital neuralgia or trigeminal neuralgia.

Therapy system 110, however, may be useful in other neurostimulation applications. Thus, in alternate embodiments, target tissue sites 118 and 119 may be at locations proximate to any other suitable nerve in body of patient 116, which may be selected based on, for example, a therapy program selected for a particular patient. For example, in other embodiments, therapy system 110 may be used to deliver neurostimulation therapy to other areas of the nervous system, in which cases, lead 114 would be implanted proximate to the respective nerve(s). As one example, leads 114 may be implanted proximate to other nerves and/or structures of the head and neck of patient 116. As another example, system 110 may be implanted at other locations in a patient and used for sacral stimulation, pelvic floor stimulation, peripheral nerve field stimulation, spinal cord stimulation, deep brain stimulation, gastric stimulation, or subcutaneous stimulation other than occipital stimulation.

Accurate lead placement may affect the success of occipital nerve stimulation. If lead 114 is located too deep, i.e., anterior, in the subcutaneous tissue, patient 116 may experience muscle contractions, grabbing sensations, or burning. Such problems may additionally occur if one of leads 114 migrates after implantation. However, because electrical stimulator 112 is located proximate to target tissue sites 118 and 119, leads may be less than six inches in length, which may provide a low electrical resistance and improve the efficiency of therapy system 110. Additionally, the short length of leads 114 also limits the potential for lead migration because patient movement does not create a significant stress on leads 114. In some embodiments, leads 114 may include fixation elements such as tines.

Therapy system 110 also may include a clinician programmer 126 and a patient programmer 128. Clinician programmer 126 may be a handheld computing device that permits a clinician to program neurostimulation therapy for patient 116, e.g., using input keys and a display. For example, using clinician programmer 126, the clinician may specify stimulation parameters for use in delivery of electrical stimulation therapy. Clinician programmer 126 supports telemetry (e.g., radio frequency telemetry) with electrical stimulator 112 to download neurostimulation parameters and, optionally, upload operational or physiological data stored by electrical stimulator 112. In this manner, the clinician may periodically interrogate electrical stimulator 112 to evaluate efficacy and, if necessary, modify the stimulation parameters.

Like clinician programmer 126, patient programmer 128 may be a handheld computing device. Patient programmer 128 may also include a display and input keys to allow patient 116 to interact with patient programmer 128 and electrical stimulator 112. In this manner, patient programmer 128 provides patient 116 with an interface for control of neurostimulation therapy by electrical stimulator 112. For example, patient 116 may use patient programmer 128 to start, stop or adjust neurostimulation therapy. In particular, patient programmer 128 may permit patient 116 to adjust stimulation parameters such as duration, amplitude, current, waveform, pulse width and pulse rate, within an adjustment range specified by the clinician via clinician programmer 128, or select from a library of stored stimulation therapy programs.

Electrical stimulator 112, clinician programmer 126, and patient programmer 128 may communicate wireless communication, as shown in FIG. 6A. Clinician programmer 126 and patient programmer 128 may, for example, communicate via wireless communication with electrical stimulator 112 using RF telemetry techniques known in the art. Clinician programmer 126 and patient programmer 128 also may communicate with each other using any of a variety of local communication techniques, such as RF communication according to the 802.11 or Bluetooth specification sets, infrared communication, e.g., according to the IrDA standard, or other standard or proprietary telemetry protocols.

In other embodiments, programmers 126 and 128 may communicate via a wired connection, such as via a serial communication cable, or via exchange of removable media, such as magnetic or optical disks, or memory cards or sticks. Further, the clinician programmer 126 may communicate with patient programmer 128 via remote telemetry techniques known in the art, communicating via a local area network (LAN), wide area network (WAN), public switched telephone network (PSTN), or cellular telephone network, for example.

FIG. 6B illustrates techniques for implantation of the therapy system 110 of FIG. 6A by a surgeon, physician, clinician or other caregiver. First, a clinician shaves the back of the head of patient 16 to ensure hair stays out of the way during the implantation. Then, as illustrated in FIG. 6B, incision 131 is made in the skin scalp of patient 116 along midline 109 of patient 116 inferior to inion 120. For example, incision 131 may start about 1 cm (e.g., a finger width) below the inion in the scalp of the patient. After incision 131 is made, lateral paths 138A and 138B (collectively referred to as “lateral paths 138”) are tunneled to both the left and the right of incision 131 for leads 114 (FIG. 6A). For example, lateral paths 118 may be formed using blunt dissection. Inferior pocket 136 is also made for electrical stimulator 112 immediately below pockets 138. The distal ends of leads 114 including paddles 117 are inserted into pockets 138. Electrical stimulator 112 is rotated 180 degrees to twist leads 114 as shown in FIG. 6A. This rotation takes up slack in leads 114 to allow leads 114 to lie flat against the skull of patient 116 after implantation. Next, electrical stimulator 112 is inserted into pocket 136 via incision 131 by the clinician. Incision 131 only needs to be large enough so that electrical stimulator 112 may fit through incision 131. Then, incision 131 is closed over the implanted leads 114 and electrical stimulator 112. For example, incision 131 may be closed using glue and a vertical mattress suture technique. Other techniques such as taping or stapling may also be used.

Optionally, the distal ends of leads 114 may be secured in place. For example, leads 114 may include tines or the distal ends of leads 114 may be secured directly with a suture. In addition the housing of electrical stimulator 112 may also be secured in place using a suture.

Alternatively, a lateral incision may be used instead of lateral paths 138. Other embodiments may comprise using a lateral incision with paddle leads or using a midline incision with leads including ring electrodes instead of paddle leads.

In all embodiments, fluoroscopy may be used to locate the leads adjacent the target sites during the implantation. Additionally, patient 116 may be located on his or her side during implantation, which would allow an anesthesiologist to see his or her face.

FIG. 7 illustrate another example of kit 227, which includes medical lead 220 and medical lead introducer 210. Medical lead introducer 210 is similar to medical lead introducer 10 except that the carrier structure of lead introducer 210 is recession 236 and the mating carrier structure of medical lead 220 is tab 238. The combination of tab 238 and recession 236 operates in a functionally similar manner to tab 16 and through-hole 26 of lead introducer 210 and lead 220 respectively. As kit 227 is inserted within a patient, recession 236 engages tab 238, and as lead introducer 210 is withdrawn, lead introducer 210 pulls free from lead 220. Except for the different carrier structure lead introducer 210 generally conforms to lead introducer 10. Likewise, except for the different mating carrier structure lead 220 generally conforms to lead 20. For brevity, details discussed with respect to medical lead introducer 10 and lead 20 that are the same for lead introducer 210 and lead 220 respectively are not repeated with respect to FIG. 7. For this reason details described with respect to medical lead introducer 10 and lead 20 should also be attributed to lead introducer 210 and lead 220.

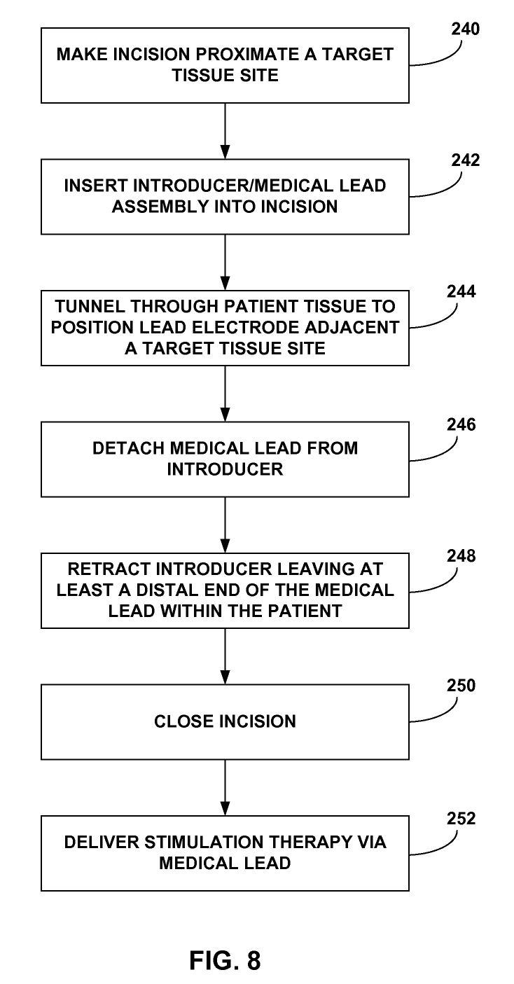

FIG. 8 is a flowchart that illustrates techniques for introducing a medical lead. For clarity, the techniques shown in FIG. 8 are described with respect to patient 116 (FIGS. 6A-6B) and medical lead introducer 10 and lead 20 (FIGS. 1A-3). As an example, lead 20 should be considered substantially to be the same as lead 114B (FIG. 6A). First, a clinician makes incision 131 in the skin of the patient proximate to target tissue site 118 (240). Next, the clinician inserts an assembly including lead introducer 10 and lead 20 into the incision (242). The assembly is then forced through the patient tissue tunneling under the scalp of patient 116 to position electrodes 25 adjacent to target tissue site 118 (244). The clinician detaches lead 20 is detached from lead introducer 10 by beginning to withdraw lead introducer 10 (246). The clinician then withdraws lead introducer 10, leaving lead 20 in place adjacent to target tissue site 118 (248).

With respect to patient 116, the process is repeated to position lead 114A adjacent to target tissue site 119. The clinician forms pocket 136 for electrical stimulator 112 below incision 136 and positions electrical stimulator 112 in pocket 136 before closing incision 131 (250). The implanted system is then used to deliver stimulation therapy to patient 116, e.g., the implanted system may configured using clinician programmer 126 and/or patient programmer 128 (252).

Various embodiments have been described. The foregoing description of the exemplary embodiments of the invention has been presented for the purposes of illustration and description. They are not intended to be exhaustive or to limit the invention to the precise forms disclosed. Many modifications and variations are possible in light of the above teaching. The scope of the invention is not limited with this detailed description, but rather by the claims. These and other embodiments are within the scope of the following claims.

1. A medical lead introducer comprising:
a shank;
a carrier structure on the shank configured to engage a mating carrier structure of a medical lead during a lead introduction procedure; and
a blunt dissection element located on a distal end of the shank,
wherein the blunt dissection element is configured to shield at least a distal portion of the medical lead when the medical lead is engaged by the carrier structure during the lead introduction procedure.

2. The medical lead introducer of claim 1, wherein the carrier structure is a tab that extends from the shank and the mating carrier structure is a depression.

3. The medical lead introducer of claim 2, wherein a width of the shank is at least three times greater than a height of the shank, and wherein the tab extends beyond the height of the shank.

4. The medical lead introducer of claim 2, wherein the shank has a C-shaped cross section including a center element and two side elements extending from the edges of the center element, wherein the tab extends from the center element in substantially the same direction as the side elements.

5. The medical lead introducer of claim 2, wherein the tab is an at least partially retractable tab.

6. The medical lead introducer of claim 1, wherein the shank has a substantially rectangular cross section.

7. The medical lead introducer of claim 1, further comprising a handle attached to a proximal end of the shank.

8. The medical lead introducer of claim 1, wherein the blunt dissection element includes a first surface proximate a side of the shank that includes the carrier structure and a second surface opposing the first surface, wherein the frontal area of the first surface is greater than the frontal area of the second surface.

9. The medical lead introducer of claim 1, wherein the blunt dissection element defines a asymmetrical shape such that the medical lead introducer is configured to balance an insertion force against a blunt dissection force to limit bending of the medical lead introducer resulting from the combination of the insertion force and the blunt dissection force.

10. The medical lead introducer of claim 1, wherein the medical lead introducer is configured such that at least one side of the medical lead is exposed to patient tissue during the lead introduction procedure.

11. A method for introducing a medical lead comprising:
inserting an assembly into a patient, the assembly comprising:
a medical lead introducer including a blunt dissection element located at a distal end of the medical lead introducer, and
a medical lead attached to the medical lead introducer;
advancing the assembly through tissue of the patient to locate at least a portion of the medical lead proximate to a target tissue site, wherein advancing the assembly causes the blunt dissection element to tunnel through tissue of the patient;
detaching the medical lead from the medical lead introducer; and
retracting the medical lead introducer leaving a stimulation electrode of the medical lead within the patient proximate to the target tissue site.

12. The method of claim 11, further comprising:
making an incision in the skin of the patient proximate to the target tissue site; and
inserting the assembly into the incision prior to advancing the assembly through tissue of the patient.

13. The method of claim 12 further comprising closing the incision.

14. The method of claim 11, wherein the blunt dissection element has a frontal area that shields at least a portion of the frontal area of the medical lead.

15. The method of claim 11, wherein the medical lead includes a paddle with electrodes at the distal end of the medical lead.

16. The method of claim 11, wherein the medical lead introducer further includes:
a shank, wherein the blunt dissection element is fixed to a distal end of the shank; and
a tab extending from the shank holding a distal end of the medical lead during the insertion,
wherein the blunt dissection element shields the medical lead from patient tissue when the medical lead is held by the tab during the insertion.

17. The method of claim 16, wherein the medical lead forms a depression, wherein the tab is configured to engage the medical lead at the depression.

18. The method of claim 16, wherein the shank has a substantially rectangular cross section.

19. The method of claim 16, wherein a width of the shank is at least three time greater than a height of the shank, wherein the tab extends beyond the height of the shank.

20. The method of claim 16, wherein the shank has a C-shaped cross section including a center element and two side elements extending from the edges of the center element, wherein the tab extend from the center element in the same direction as the side elements.

21. The method of claim 16, wherein the medical lead introducer includes a handle on the proximal end of the medical lead introducer, wherein advancing the assembly through tissue of the patient comprises pushing on the handle.

22. The method of claim 11, wherein the blunt dissection element is asymmetrical such that the medical lead introducer is configured to balance an insertion force against a blunt dissection force to limit bending of the medical lead introducer resulting from the combination of the insertion force and the blunt dissection force.

23. The method of claim 11, wherein the medical lead introducer is configured such that at least one side of the medical lead is exposed to patient tissue during the insertion.

24. The method of claim 11, wherein the target tissue site includes at least one of a group consisting of:
a trigeminal nerve;
a greater occipital nerve;
a lesser occipital nerve;
a third occipital nerve; and
a suboccipital nerve.

25. A kit to facilitate implantation of a medical lead into a patient comprising:
the medical lead; and
a medical lead introducer comprising:
a shank;
a carrier structure on the shank configured to engage a mating carrier structure of the medical lead during a lead introduction procedure; and
a blunt dissection element located on a distal end of the shank,
wherein the blunt dissection element is configured to shield the medical lead when the medical lead is held by the carrier structure during a lead introduction procedure.

26. The kit of claim 25, wherein the blunt dissection element has a frontal area that extends beyond a frontal area of the shank to shield at least a portion of the frontal area of the medical lead during the lead introduction procedure.

27. The kit of claim 25, wherein the shank has a rectangular cross section.

28. The kit of claim 25, wherein the carrier structure is a tab that extends from the shank and the mating carrier structure is a depression.

29. The kit of claim 28, wherein a width of the shank is at least three time greater than a height of the shank, wherein the tab extends beyond the height of the shank.

30. The kit of claim 28, wherein the shank has a C-shaped cross section including a center element and two side elements extending from the edges of the center element, wherein the tab extend from the center element in the same direction as the side elements.

31. The kit of claim 28, wherein the tab is a retractable tab.

32. The kit of claim 28, wherein the depression at the distal end of the medical lead is a through-hole.

33. The kit of claim 25, further comprising a handle attached to a proximal end of the shank.

34. The kit of claim 25, wherein the blunt dissection element includes a first surface proximate a side the shank that includes the carrier structure and a second surface opposing the first surface, wherein the frontal area of the first surface is less than the frontal area of the second surface.

35. The kit of claim 25, wherein the blunt dissection element is asymmetrical such that the medical lead introducer is configured to balance an insertion force against a blunt dissection force to limit bending of the medical lead introducer resulting from the combination of the insertion force and the blunt dissection force.

36. The kit of claim 25, wherein the medical lead introducer is configured such that at least one side of the medical lead is exposed to patient tissue during the lead introduction procedure.

37. The kit of claim 25, wherein the medical lead includes a paddle with electrodes at the distal end of the medical lead and is in the paddle.

38. The kit of claim 25, further comprising a sterile container containing the medical lead introducer and the medical lead.
